(19) United States
(12) Patent Application Publication
(10) Pub. No.: US 2008/0104719 A1
(43) Pub. Date: May 1, 2008

(54) CISD2-Knockout Mice and Uses Thereof

(75) Inventors: Ting-Fen Tsai, Taipei (TW); Yi-Fan Chen, Taipei (TW); Shih-Feng Tsai, Miaoli County (TW); Ya-Ting Chen, Miaoli County (TW)

Correspondence Address: WPAT, PC;INTELLECTUAL PROPERTY ATTORNEYS, 2030 MAIN STREET, SUITE 1300, IRVINE, CA, 92614, US

(73) Assignees: NATIONAL YANG-MING UNIVERSITY, Taipei City (TW); NATIONAL HEALTH RESEARCH INSTITUTES, Miaoli County (TW)

(21) Appl. No.: 11/866,374

(22) Filed: Oct. 2, 2007

Related U.S. Application Data
(63) Continuation of application No. 60/849,089, filed on Oct. 3, 2006.

(51) Int. Cl. A01K 67/027; C12N 5/00; C12Q 1/68; G01N 33/53

(52) U.S. Cl. 800/3; 435/325; 435/6; 800/18

(57) ABSTRACT

An aging animal model and a method for screening an agent for treating or preventing aging associated phenotypes or delaying onset of aging.

### CROSS-REFERENCE TO RELATED APPLICATIONS

The present invention claims priority to U.S. Application No. 60/849,089, filed Oct. 3, 2006, which is incorporated herein by reference in its entirety.

### FIELD OF THE INVENTION

The present invention relates to an aging animal model and use thereof.

### BACKGROUND OF THE INVENTION

Aging, or organismal senescence, is defined as gradual changes in an organism that “adversely affect its vitality and function, but most importantly, increases the mortality rate of an organism as a function of time”.

Aging can be characterized as the age-related decline of physiological functions necessary for the survival and reproduction of an organism. Common age-associated diseases connected to these functions include, but not limited to, arteriosclerosis, cancer, dementia and osteoporosis. To understand the primary causes of these diseases' onset and commencing of generalized malfunctions of multiple organ systems that potentially shorten life span and reduce fertility is central to understanding human aging.

A number of genetic components of aging have been identified using model organisms, ranging from the bakers' yeast (Saccharomyces cerevisiae), the soil roundworm (Caenorhabditis elegans), the fruit fly (Drosophila melanogaster), and the mouse (Mus musculus).

In yeast, Sir2 is required for genomic silencing at three loci: the yeast mating loci, the telomeres and the ribosomal DNA (rDNA). In some species of yeast replicative aging may be partially caused by homologous recombination between rDNA repeats; excision of rDNA repeats results in the formation of extrachromosomal rDNA circles (ERCs). These ERCs replicate and preferentially segregate to the mother cell during cell division, and are believed to result in cellular senescence by titrating away (competing for) essential nuclear factors. ERCs have not been observed in other species of yeast (which also display replicative senescence), and ERCs are not believed to contribute to aging in higher organisms such as humans. Extrachromosomal circular DNA (eccDNA) has been found in worms, flies and humans. The role of eccDNA in aging, if any, is unknown. In yeast, Sir2 activity is regulated by the nicotinamidase PNC1. PNC1 is transcriptionally upregulated under stressful conditions such as caloric restriction, heat shock, and osmotic shock. By converting nicotinamide to niacin, it removes nicotinamide, which inhibits the activity of Sir2.

C. elegans is also a powerful model system for the study of aging, because of its genetics, relatively short life span, and ease of propagation of populations of synchronized individuals. Numerous single-gene mutations (AGE genes) have been identified that increase C. elegans life span. The best characterized of these (daf-2, age-1) are in an insulin-like signaling pathway which culminates in altering the activity of the transcription factor daf-16. This same signaling pathway controls the entry of developing nematodes into the alternate, dauer larval stage. The cost to fitness of these longevity mutants predicted by evolutionary theory was observed under stressful laboratory conditions.

One approach to understanding the molecular basis of human aging is to find genes that determine inherited premature aging syndromes thereby causing rapid development of these senescence associated diseases early in life. To that end, mutant mice that display multiple phenotypes resembling accelerated aging have been developed in recent years. However, virtually all of them display partial spectrum of the senescence associated phenotypes.

### SUMMARY OF THE INVENTION

The present invention provides a knockout mouse whose genome is disrupted by an inserting sequence or mutant at Cisd2 gene locus so as to produce a phonotype, relative to a wild-type phenotype.

The present invention also provides a cell or cell line which comprises a targeted disruption in Cisd2 gene in which Cisd2 exon 3 has been disrupted.

The present invention further provides a method for screening a candidate agent for preventing or treating aging associated phenotypes or delaying onset of aging comprising: (a) providing the mouse of the present invention, the cell or cell line of the present invention; (b) adding to said candidate agent, and (c) determining the agent by identifying the desired therapeutic effects in ameliorating aging associated phenotype.

### BRIEF DESCRIPTION OF THE DRAWINGS

To adequately describe the present invention, references to embodiments thereof are illustrated in the appended drawings. These drawings herewith form a part of the specification. However, the appended drawings are not to be considered limiting in their scope.

FIG. 1a shows genes and markers located in the human chromosome 4q23-4q25 region, to which the human longevity locus was previously linked and was deemed to be near marker D4S1564. ZCD2, the human ortholog of the mouse gene targeted in the present invention, is located between the ubiquitin-conjugating enzyme E2D 3 (UBE2D3) and 3-hydroxybutyrate dehydrogenase type 2 (BDH2) genes illustrated in FIG. 1a.

FIG. 1b shows Northern blot analysis of Cisd2 mRNA expression in mouse tissues. A corresponding 18S rRNA band for each lane is used to assist in normalizing the band intensity on the Northern blot.

FIG. 2a shows the genomic structure of the wild-type and the resulting targeted alleles of the Cisd2 gene. The Cisd2 gene was disrupted by a targeted insertion vector containing puromycin (Puro) selection cassette. A probe used to identify the targeted events by Southern blot analysis is indicated along with the diagnostic EcoRI sites in the Cisd2 gene and the replacement region from the targeting construct.

FIG. 2b shows Southern blot hybridization of tail DNA isolated from wild-type (+/+), heterozygous (+/−) and homozygous (−/−) offspring of a heterozygous intercross using a 3′ flanking probe.

FIG. 2c shows Northern blot analysis of Cisd2 mRNA isolated from brain tissues of 2-month old wild-type (+/+), heterozygous (+/−) and homozygous (−/−) offspring.

FIG. 2d illustrates pedigree of three generations of mice carrying the Cisd2 mutant allele, with mice grouped by sex and genotype. Square, male; circle, female; checkered symbol, chimera; open symbols, Cisd2 wild-type; filled symbols, Cisd2 homozygous (−/−) knockout; half filled symbols, Cisd2 heterozygous (+/−) knockout.

FIG. 3a shows onset of age-related phenotypes as a function of age by Cisd2 homozygous (−/−) knockout mice. These mice exhibit severe growth retardation and premature aging phenotypes.

FIG. 3b shows survival rate of Cisd2 homozygous mice. The percent survival of wild-type (+/+, n=10), heterozygous (+/−, n=16) and homozygous (−/−, n=16) mice including males and females is plotted against the age in months.

FIG. 3c shows growth curve of male and female mice with different genotypes. Body weight is plotted against age of mice in weeks.

FIG. 4a shows premature aging related symptoms, including hair graying, protruding ears, and prominent eyes, in 12-month-old Cisd2.sup.−/− mice.

FIG. 4b shows 6-month-old Cisd2.sup.−/− mice develop blindness.

FIG. 4c shows opacity of cornea analyzed by histological examination. The H&E stain indicated collagen deposition in the lesion outside the cornea in Cisd2.sup.−/− mice.

FIG. 4d shows early depigmentation of the fur in 12-month-old Cisd2.sup.−/− (−/−) mice.

FIG. 4e shows hair follicle atrophy visualized by Masson's trichrome staining in 12-month-old Cisd2.sup.−/− mice (−/−).

FIG. 4f shows reduced percentage of hair follicle with hair in 12-month-old Cisd2.sup.−/− mice (−/−) relative to that for age-matched heterozygous mice (+/−) and wild-type (+/+).

FIGS. 5a and 5b show cross sections of skin from 12-month-old Cisd2.sup.+/+ and age-matched Cisd2.sup.−/− mice, respectively.

FIG. 5c shows graphs comparing mean thickness of subcutaneous muscle, adipose layer and dermis between 12-month-old Cisd2.sup.+/+ (+/+) and age-matched Cisd2.sup.−/− (−/−) mice. Asterisks indicate p<0.05 was statistically significant.

FIG. 6a shows micro-computed tomography imaging of the trabeculae in the femur of 4-month-old wild-type (+/+) and age-matched homozygous (−/−) mice.

FIG. 6b shows femur density of wild-type (+/+), heterozygotes (+/−), and homozygotes (−/−), was analyzed by dual energy x-ray absorpitometer (DEXA).

FIG. 6c shows whole-body radiography of a 4-month-old wild-type (+/+) and homozygous (−/−) mouse.

FIG. 6d shows micro-computed tomography scanning for 3D reconstruction of thoracic and spinal columns of a 5-month-old wild-type (+/+) and an age-matched homozygous (−/−) mouse.

FIG. 6e shows a decrease of mean thoracic volume in a homozygous (−/−) versus an age-matched wild-type (+/+) mouse.

FIGS. 6f and 6g compare 2 respiratory parameters, mean tidal volume and enhanced pause, respectively, between various age-matched homozygous (−/−) vs. wild-type (+/+) mice, *p<0.05; **p<0.005.

FIG. 7a shows a photograph of skinned 20-month-old heterozygous (+/−) and homozygous (−/−) mice. No detectable phenotypic difference was observed with respect to muscle and body fat for the wild-type (+/+) and heterozygous (+/−) mice. FIG. 7b shows quantification of body fat by collecting and weighting all of the fat from the whole body.

FIGS. 7c and 7d show H&E staining of longitudinal and transverse sections of muscle, respectively, prepared from a representative aged (28-month old) wild-type (+/+) mouse. Arrows indicate degenerated muscle fibers in the aged mice.

FIGS. 7e and 7f show H&E staining of longitudinal and transverse sections of muscle, respectively, prepared from a representative 2-month-old Cisd2 homozygous (−/−) mouse.

FIGS. 7g and 7h show H&E staining of longitudinal and transverse sections of muscle, respectively, prepared from a representative 2-month-old wild-type (+/+) mouse.

FIG. 8a shows an electron micrograph of section of muscle from a wild-type (+/+) mouse.

FIG. 8b shows a similarly prepared section of muscle from a homozygous (−/−) mouse.

FIG. 8c shows an electron micrograph of the degenerated margin of striated muscle cell.

FIG. 8d shows the degenerated margin of injury striated muscle cell. The debris (D) of muscle cell and degenerated myofilaments (arrows) were separated from muscle cell. Myf, myofibril; N, nucleus; M, mitochondrion; V, digestive vacuole; Ly, lysosome.

FIGS. 9a and 9b show a transversely sectioned myelinated nerve fiber from the peripheral nerves of skeletal muscle of wild-type and Cisd2.sup.−/− mice, respectively. The axon is enveloped by the myelin sheath (MS) formed by fusing many layers of Schwann cell plasma membrane. Myelin sheath degeneration, highlighted by asterisks, was detected only in the Cisd2.sup.−/− mice.

FIG. 9c shows RT-PCR analysis of BDNF, NT-3 and TrkB mRNA isolated from brain of 3-month old wild-type and Cisd2 homozygous mice. Hprt and Actb are used as internal controls. BDNF, brain-derived neurotrophin factor; NT, neurotrophin, Trk, tyrosine receptor kinase; Hprt, Hypoxanthine guanine phosphoribosyl transferase; Actb, beta-actin.

FIG. 9d shows relative quantification by real-time PCR of BDNF mRNA isolated from the brain of various ages of wild-type mice (gray bars) and different genotypes of the Cisd2 knockout mice (black bars).

### DETAILED DESCRIPTION OF THE INVENTION

### Definition

The Cisd2-knockout mouse used in the present invention is equal to the ZCD2-knockout mouse in U.S. Application No. 60/849,089.

The term “Cisd2” as used herein means Mus musculus CDGSH iron sulfur domain 2, and the orthologous genes including ZCD2, Miner1, Noxp70, AI848398, 1500009M05Rik, 1500026J14Rik, 1500031D15Rik, and B630006A20Rik.

The present invention recapitulates a more extensive set of early senescence associated features of human aging than those previously described. As such, the present invention provides an extremely useful model to elucidate aging in human.

Furthermore, the present invention offers an in vivo system to screen for agents in ameliorating the patho-physiological effects of aging.

A mutant animal of the present invention can be any non-human mammal, preferably a mouse. A mutant animal can also be, for example, any other non-human mammals, such as rat, rabbit, goat, pig, dog, cow, or a non-human primate. It is understood that mutant animals having a disrupted Cisd2 gene, as disclosed herein, or other mutant forms that eliminate the expression of Cisd2, can be used in methods of the invention. Thus, the mutant animal loss of all or a part of the Cisd2 gene function is due to a disruption of the Cisd2 gene

The present invention provides a line of genetically engineered mice either heterozygous (referred to as Cisd2.sup.+/−) or homozygous (referred to as Cisd2.sup.−/−) for the disrupted endogenous Cisd2 gene. This gene may be mutated by disrupting one or more of its exons by heterologous DNA sequences such as an HPRT cassette using standard molecular biological techniques. In addition, any mutant forms that eliminate the expression of Cisd2 can be used. The resulting Cisd2.sup.−/− mice exhibit a range of phenotypes similar to those of human aging including many physical or biochemical manifestations as detailed below. As such, these mice, Cisd2.sup.+/− and Cisd2.sup.−/− included, can be used as a model system to help delineate the molecular mechanisms underlying human aging.

The present invention also provides a cell or cell line from the Cisd2 knockout mouse, wherein the cell or cell line contains a targeted disruption in Cisd2 gene in which Cisd2 exon 3 has been disrupted. The cell or cell line is an undifferentiated cell which is selected from the group consisting of: a stem cell, embryonic stem cell oocyte and embryonic cell.

The present invention further demonstrates a method of screening for agents useful in treating or preventing aging associated phenotypes or delaying the onset of aging consisting of administering candidate compounds to the Cisd2.sup.−/− mice or the cell or cell line derived from the Cisd2.sup.−/− and screening for the desired therapeutic effects.

The method for identifying a target gene having altered expression in a mutant Cisd2 mouse involves comparing the expression of one or more genes in a mutant mouse having a disrupted Cisd2 gene with the expression of said one or more genes in a wild type animal to identify a gene having altered expression in said mutant mouse, thereby identifying a target gene having altered expression in a mutant Cisd2 mouse.

As described in Example 7, Cisd2 mutant mice exhibited altered expression of genes in comparison to wild type mice in addition to other phenotypes described. For instance, Cisd2 knockout mice are characterized by decreased expression of BDNF. The altered expression of BDNF gene, as well as other genes having altered expression in a mutant Cisd2 mouse, indicates that Cisd2 normally regulates the expression of these genes in wild-type mice. Thus, these represent genes that can be modulated to reverse, or at least partially reverse, the physiological and biochemical characteristics of a Cisd2.sup.−/− phenotype. For example, restoring the expression of one of these Cisd2 regulated genes having altered expression in a mutant Cisd2 mouse to a level that can result in reversed phenotypes can be contemplated. Therefore, a compound that exhibits the said effect is a potentially useful therapeutic compound for treatment of aging associated phenotypes or possibly delaying the onset of aging.

As such, the present invention provides methods for identifying target genes having altered expression in a mutant Cisd2 mouse, as well as methods for identifying a compound that restores a target gene having altered expression in a mutant Cisd2 mouse to a level of expression achieving the desired therapeutic effect.

The methods of the invention for identifying a target gene having altered expression in a mutant Cisd2 mouse can involve comparing the expression of one or more genes contained within one or more organs of the mutant Cisd2 mice.

The method for identifying a compound that restores a target gene having altered expression in a mutant Cisd2 mouse to a therapeutic level of expression involves (a) contacting a target gene having altered expression in a mutant Cisd2 mouse with a test compound; (b) determining expression of said target gene, and (c) identifying a compound that modulates expression of said target gene to a level of expression consistent with a wild type level of expression.

The methods of the invention for screening for a compound that restores a target gene having altered expression in a mutant Cisd2 mouse to a more normal level of expression-involve contacting a sample exhibiting altered expression of a target gene characteristic of a mutant Cisd2 mouse with a test compound. A test compound can be any substance, molecule, compound, mixture of molecules or compounds, or any other composition which is suspected of being capable of restoring an expression level of a target gene to a more normal level.

Additionally, a test compound can be pre-selected based on a variety of criteria. For example, suitable test compounds having known modulating activity on a pathway suspected to be involved in a mutant Cisd2 phenotype can be selected for testing in the screening methods. Alternatively, the test compounds can be selected randomly and tested by the screening methods of the present invention.

A level of protein expression corresponding to a gene expression level also can be determined, if desired. A variety of methods well known in the art can be used to determine protein levels either directly or indirectly.

The methods of the invention for identifying a compound that restores a target gene having altered expression in a mutant Cisd2 mouse to a more normal level of expression can involve determining an activity of a target gene. The activity of a molecule can be determined using a variety of assays appropriate for the particular target. A detectable function of a target gene can be determined based on known or inferred characteristics of the target gene.

For use as a therapeutic agent, the compound can be formulated with a pharmaceutically acceptable carrier to produce a pharmaceutical composition, which can be administered to the individual, which can be a human or other mammal.

The methods of the invention can advantageously use cells isolated from a homozygous or heterozygous Cisd2 mutant mouse for a desired purpose. For example, these cells can be used as an in vitro method to screen agents for treating or preventing aging associated phenotypes or the onset of aging. In such a method, a compound is contacted with a cell having disrupted Cisd2 expression, and screen for modulation of the target gene as described above.

Thus, the invention provides methods of screening a large number of compounds using a cell-based assay, for example, using high throughput screening, as well as methods of further testing compounds as therapeutic agents in an animal model using the Cisd2 mutant mice.

The present invention is further directed to cell lines derived from the Cisd2.sup.+/−, or Cisd2.sup.−/− mice. These cell lines are useful in studying senescence at the cellular level and in drug screening assays. Cell lines derived from the brain, kidney, lung, stomach, intestine, spleen, heart, adipose, heart and liver tissues are especially useful in these applications.

The following examples are given for the purpose of illustrating various embodiments of the invention and are not meant to limit the present invention in any fashion.

### EXAMPLE 1

### Expression Analysis of the Mouse Cisd2 Gene

The mouse Cisd2 (SEQ ID NO. 1) was identified as the putative ortholog based on the remarkable protein sequence similarity (96% identity) to the human gene, ZCD2, located in the region where the longevity locus was previously mapped. It was then engineered and disrupted to understand its role in longevity in the present invention.

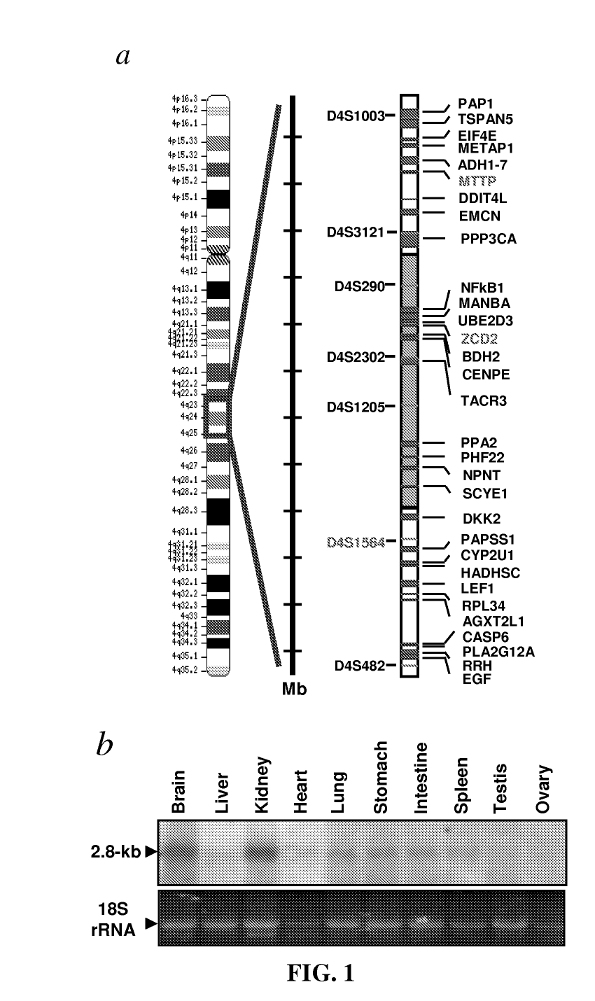

The expression pattern of the Cisd2 gene was characterized by examining the relative levels of mRNA present in adult mouse tissues (FIG. 1b). A band of 2.8-kb was detected at higher levels in brain and kidney. A similarly sized band but at lower levels was detected in lung, stomach, intestine, and spleen. There were much lower levels in liver, heart, testis and ovary.

### EXAMPLE 2

### Generation of Cisd2.sup.+/− and Cisd2.sup.−/− Mice

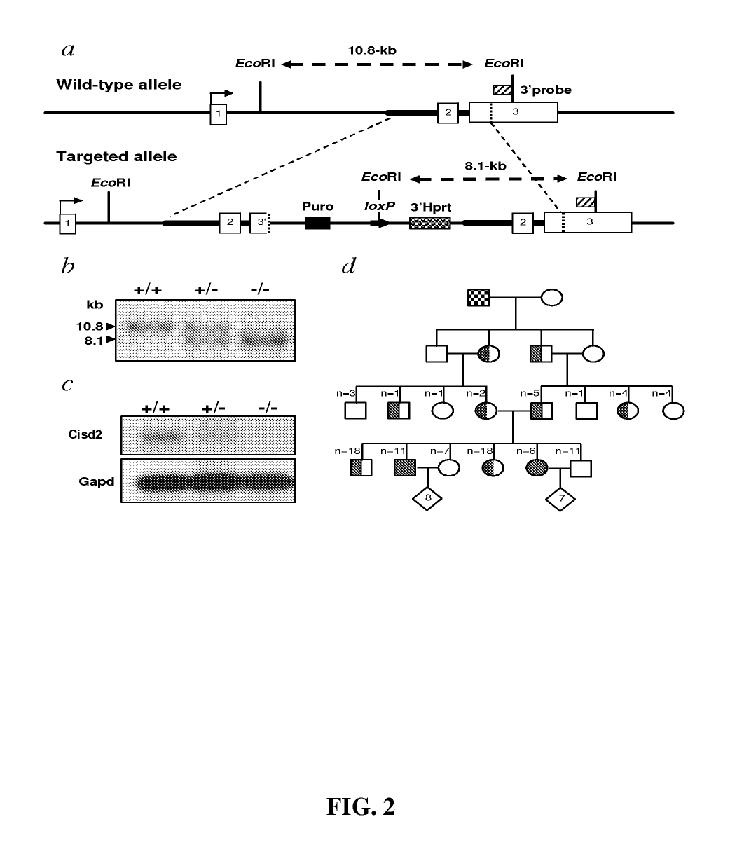

FIG. 2a shows the strategy used to create the targeted mutation. A BAC library (Research Genetics Inc.) derived from mouse strain C57BL/6 genomic DNA was screened with two pair of primers (SEQ ID NO. 2-5) designed from conserved sequence of Cisd2 gene between human (Hs.29835) and mouse (Mm.41365). The mouse BAC clones, YM-BAC-210J1 and YM-BAC-412J13, were identified and purified. Several genomic DNA fragments covering the mouse Cisd2 gene were subcloned from YM-BAC-210J1. A SpeI-BamHI 6.4 kb DNA fragment, which contains part of intron 1, exon 2 and part of exon 3, was further subcloned and used as homologous recombination arm for construction of an “insertion-type” targeting vector for the mouse Cisd2 gene. The SpeI-BamHI 6.4 kb fragment was inserted into the EcoRV site of the pG12 vector, which contains the puromycin selection cassette, a loxP site and 5′ truncated Hprt gene. The Cisd2 targeting vector, pG12/ApaI(−)-SpB6.4, was amplified and linearized within the homologous recombination arm using ApaI.

The linearized targeting vector was electroporated into 129/SvEv embryonic stem (ES) cells. Selection medium containing puromycin and gancycloviour was applied 24 h after electroporation and maintained for 7 days. Resistant colonies were selected and re-seeded onto the feeder layer in a 96-well plate. DNA extracted from individual ES clone was isolated and detected by Southern blot analysis. The 3′ flanking probe used is a 1.7 kb BamHI-EcoRI fragment from exon3 (FIG. 2A).

The targeted ES cells were injected into C57BL/6 blastcysts and reimplanted into pseudopregnant female mice. Chimeric male mice were bred with C57BL/6 female. Genomic DNA was isolated from tail samples of the appropriate agouti progeny using proteinase K/SDS digestion and phenol/chloroform extraction method. Isolated DNA samples were further analyzed by Southern blot for germline transmission. The analysis confirmed the presence of both the endogenous and the disrupted alleles in the F1 heterozygotes. The heterozygous mice were intercrossed, and their offspring were genotyped.

Genotypes of offspring from heterozygous breeding demonstrated normal Mendelian ratios of homozygous (−/−), heterozygous (+/−) and wild-type (+/+). Fertility test of the Cisd2.sup.−/− males and females exhibited normal reproductive capability.

Southern blot analysis showed that the genomic DNA digested with EcoRI and hybridized with a probe shown in FIG. 2a gave the signals expected from the wild-type (+/+), heterozygous (+/−), and homozygous-null (−/−) animals (FIG. 2b).

Northern blot of total RNA prepared from the brain tissue of wild-type (+/+), heterozygous (+/−), and Cisd2-null (−/−) mice was probed with the .sup.32P-labeled fragment identical to that used in Southern blot analysis. The probe detected a 2.8-kbp RNA band in samples from the wild-type and heterozygous but not from the homozygous animals. Hybridization of the same filter, after stripping of the Cisd2 probe, with a mouse glyceraldehyde-3-phosphate dehydrogenase (Gapd) probe confirmed that equal amounts of RNA were loaded on the gel.

### EXAMPLE 3

### Early Senescence Including Reduced Life Span and Growth Retardation in Cisd2.sup.−/− Mice

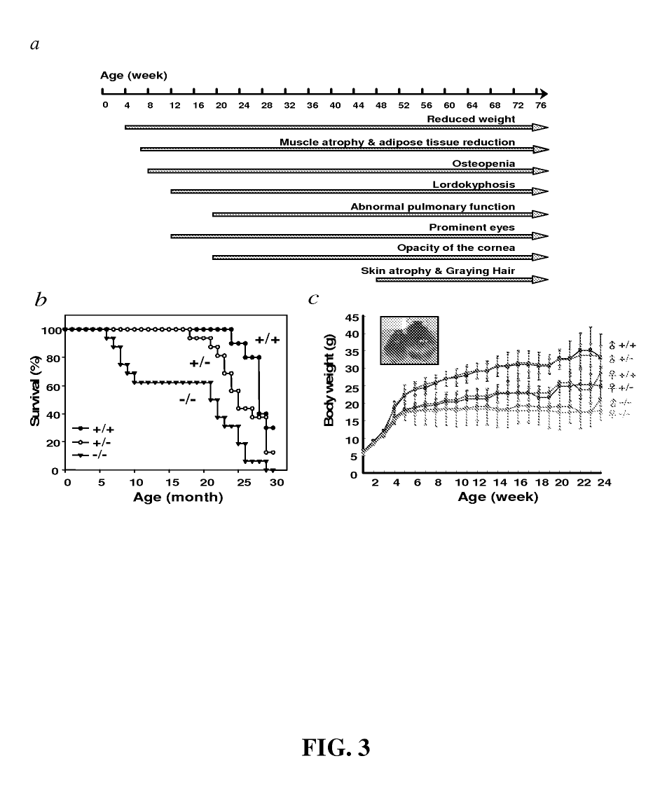

Up to 3 months of age, Cisd2.sup.−/− mice appear morphologically identical to their Cisd2.sup.+/+ littermates. However, starting around month 4, all of the Cisd2.sup.−/− mice start to display a wide range of senescence associated phenotypes shown in FIG. 3a with the time of onset indicated for each phenotype. Not surprisingly, early senescence is accompanied by shortened life span when survival of a litter is examined (FIG. 3b) and there appears to be signs of haploinsufficiency for Cisd2 in view of the slightly lower survival rate for the Cisd2.sup.+/− mice. Furthermore, growth retardation is already evident at month 4 in Cisd2.sup.−/− males and it appears there is little growth for Cisd2.sup.−/− mice after month 5 (FIG. 3c).

### EXAMPLE 4

### Eye and Cutaneous Phenotypes in Cisd2.sup.−/− Mice

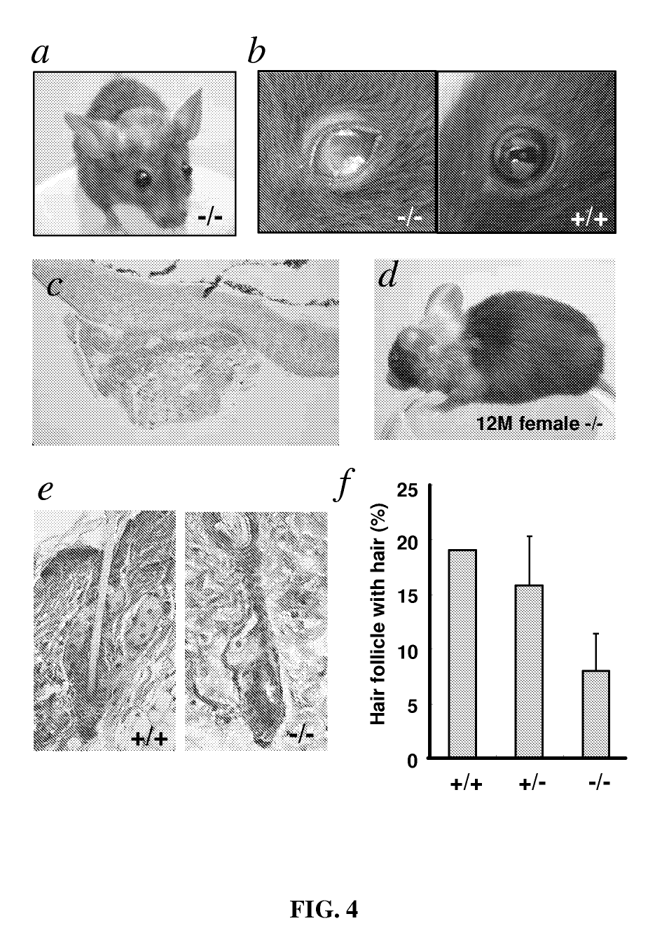

Starting in week 12, the Cisd2.sup.−/− mice begin to acquire a set of aged appearance remarkably similar to those displayed by patients with Hutchinson-Guilford progeria syndrome. These include prominent eyes and protruding ears and scalp alopecia (FIGS. 4a, e, and f). There is also early depigmentation in the fur (FIG. 4d) of 12-month-old Cisd2.sup.−/− mice where no depigmentation was observed in the aged matched Cisd2.sup.+/+ littermates. Ocular abnormalities were also observed where the 6-month-old Cisd2.sup.−/− mice had opaque eyes similar to symptoms of cataracts and became blind with accompanying cornea atrophy (FIG. 4b). The opacity of the cornea was investigated and histological analysis found collagen deposition that appears to correlate with the observed ocular phenotype.

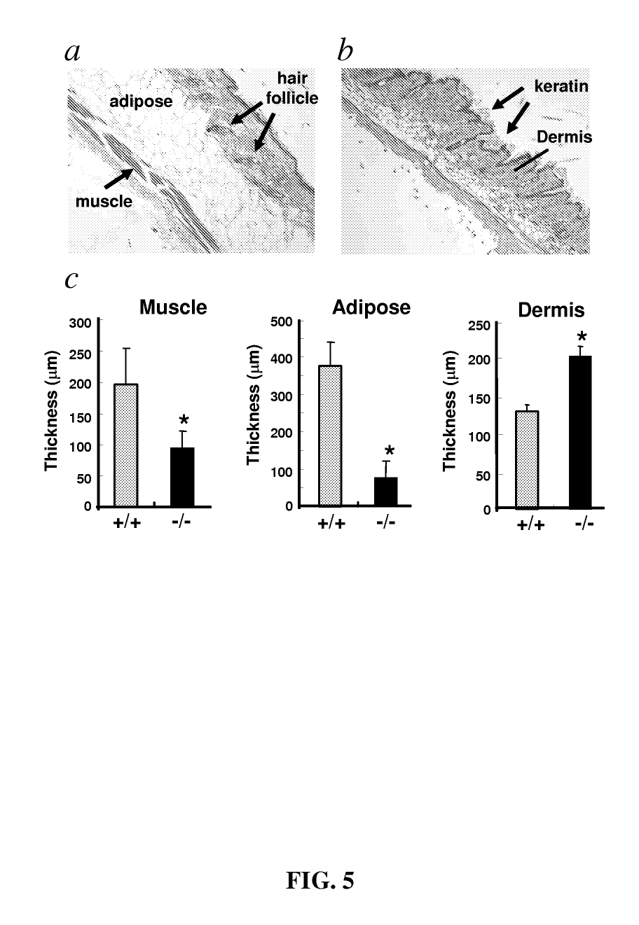

Two anatomical characteristics commonly seen in aged human skins are reduced dermal thickness and subcutaneous adipose. Consistent with those features in human, the skin of 12-month-old Cisd2.sup.−/− mice exhibits phenotypes of massive hyperkeratosis, significant decrease of subcutaneous fat and muscle, and noticeably thickened dermis with expanded surface (FIG. 5b) compared with those of age-matched wild-type mice (FIG. 5a). Though abundantly present in skin of 12-month-old wild-type mice, subcutaneous adipose cells are nearly absent in that of age-matched Cisd2.sup.−/− mice. Quantitative analysis confirmed mean thicknesses of muscle and adipose layer for skin of 12-month-old Cisd2.sup.−/− mice is considerably reduced compared with those for skin of age-matched wild-type mice while there is a concomitant increase in the mean thickness of the dermis layer.

### EXAMPLE 5

### Abnormal Skeleton and Pulmonary Functions in Cisd2.sup.−/− Mice

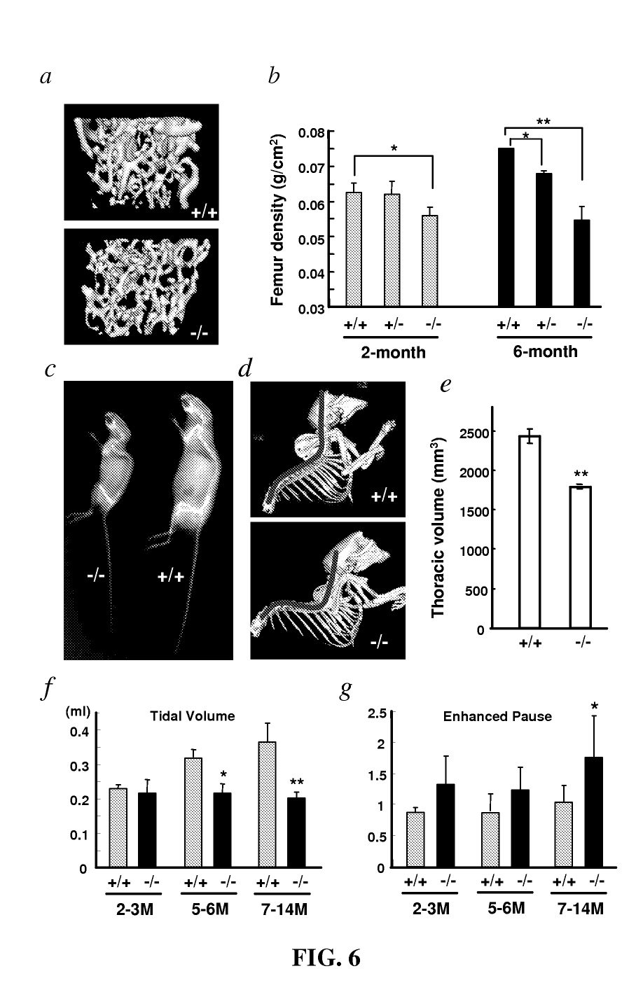

Micro-computer tomography analysis detected a decrease of femur density in the 2-month-old Cisd2.sup.−/− mice compared with that of the age-match wild-type mice while the trabeculae of the femur in Cisd2.sup.−/− mice are noticeably thinner (FIG. 6a). Interestingly, a decrease of femur density started to emerge in 6-month-old Cisd2.sup.+/− mice while a progressively more severe phenotype was observed in the age-matched Cisd2.sup.−/− mice (FIG. 6b). This shows, in addition to what was observed in life span evaluation, an apparent Cisd2 with respect to femur density but is only obvious after 6 month of age.

The bone samples of wild-type and Cisd2.sup.−/− mice were fixed in 10% buffered formalin phosphate, stored in 70% ethanol and examined by eXplore Locus SP Pre-Clinical Specimen MicroCT (GE Healthcare). Whole-body and femur scans were performed in the axial plane mounted in a cylindrical sample holder. The three-dimensional images of bones reconstructed from MicroCT scanning slices used to qualitatively evaluate bone structure and morphology. The quantitative data of bone tissue were separated from those for marrow and soft tissue and were analyzed by eXplore MicroView v. 2.0 Software Guide (GE Healthcare).

While showing no detectable skeletal abnormalities up to 2 month of age, radiographs of 4-month-old Cisd2.sup.−/− mice already displayed significant lordokyphosis (curvature of the spinal column) (FIG. 6c), which resulted in a decrease in mean thoracic volume for them compared with that for the age-matched wild-type mice (FIG. 6d and FIG. 6e). Consequently, the skeletal abnormality affects various respiratory parameters as measured by plethysmography (FIG. 6f and FIG. 6g) and leads to abnormal pulmonary functions. These features, including decrease in femur density and lordokyphosis, are manifested in aged humans.

Respiratory parameters were measured in conscious mice with three genotypes by using plethysmography chambers where the mouse body was enclosed in a sealed chamber while the head was free. Thoracic movements were measured by pressure transducers that were linked to a Buxco amplifier system and respiratory parameters, then captured and analyzed by the Notocord HEM data acquisition system. Upon placement of the mice into the plethysmography chambers, tidal volume (TV) was determined 10 min at unrestrained condition. The formula for calculating Penh (Enhanced Pause) is: PEF/PIF×(Te/Rt-1), Where Te=Expiratory time, Rt=Relaxation time, PEF is Peak Expiratory Flow, and PIF is Peak Inspiratory Flow.

### EXAMPLE 6

### Muscle Atrophy and Loss of Adipose Tissue in Cisd2.sup.−/− Mice

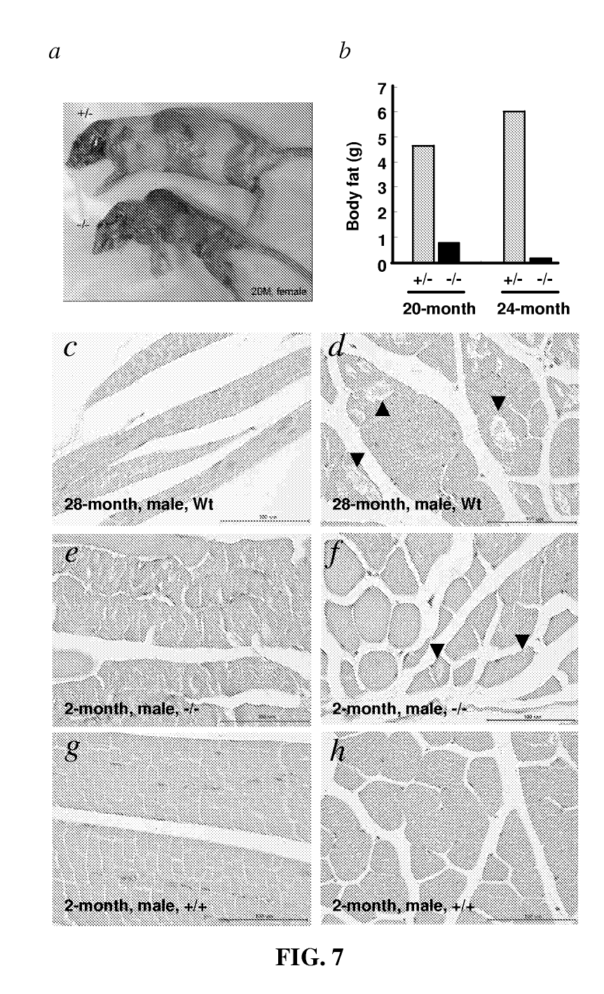

As early as week 5, muscle atrophy and reduction of adipose tissue started to take place in Cisd2.sup.−/− mice. By month 20, Cisd2.sup.−/− mice showed pronounced appearance of weight loss, lordokyphosis, and loss of adipose tissue compared with those of Cisd2.sup.+/− littermates (FIG. 7a). At month 24, Cisd2.sup.−/− mice showed a near complete loss of body fat (FIG. 7b).

The observation of muscle atrophy prompted a detailed examination of the structure and integrity of muscle fibers. Tissues were fixed in 10% buffer formalin phosphate. After embedded in paraffin, 3-4 μm tissues were transversely sectioned and stained with H&E. Sections were examined and photographed under light microscopy. Abnormally wave-shaped longitudinal fibers (FIG. 7e) were observed in 2-month-old Cisd2.sup.−/− mice, which are distinct from those in 28-month-old wild-type mice (FIG. 7c) where muscle fibers are reduced in width while structurally intact without any wavy fibers. On the other hand, there are similarly degenerated transverse fibers (arrows) present in both 28-month-old wild-type and 2-month-old Cisd2.sup. (FIG. 7d and FIG. 7f).

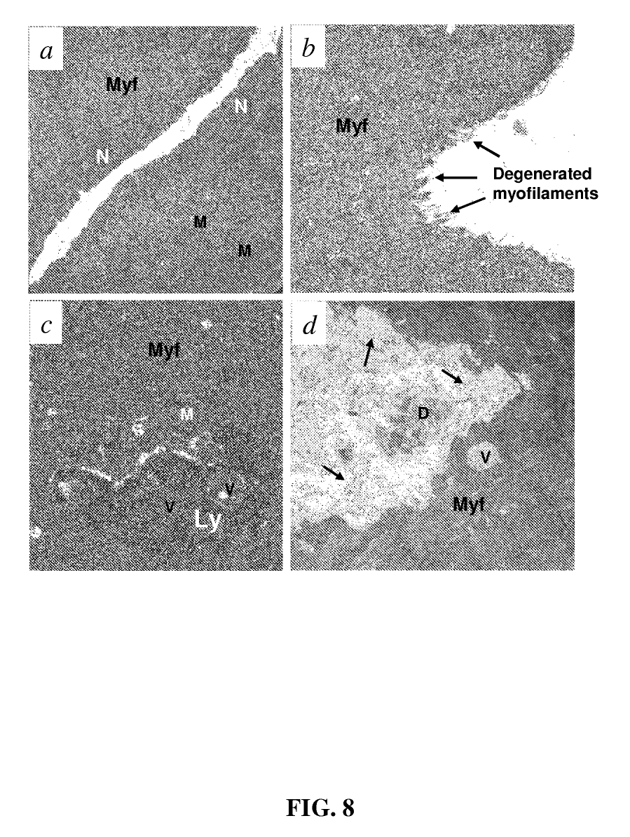

To understand the basis for the morphological abnormality in the longitudinal fibers in Cisd2.sup.−/− mice, ultrastructure of muscle cells were examined by electron microscopy. Muscle tissues from wild-type and Cisd2.sup.−/− mice were fixed in a mixture of glutaraldehyde (1.5%) and paraformaldehyde (1.5%) in phosphate buffer at pH 7.3. These were postfixed in 1% OsO4, 1.5% potassium hexanoferrate, rinsed in cacodylate and 0.2 M sodium maleate buffers (pH 6.0), and block-stained with 1% uranyl acetate. Following dehydration, tissues were embedded in Epon and were ready for transmission electron microscopy. Degenerated myofilaments indicated by arrows (FIG. 8b) were separated from a muscle cell in 2-month-old Cisd2.sup.−/− mice while myofilaments remain intact in age-matched Cisd2.sup.+/+ mice (FIG. 8a). Myofibrils of a striated muscle cell were engulfed by lysosomes. There were many digestive vacuoles in lysosome (FIG. 8c and FIG. 8d). Consistent with other phenotypes observed, both muscle atrophy and loss of adipose tissues are hallmarks of human aging.

### EXAMPLE 7

### Myelin Sheath and Axon Degeneration and Reduction of Brain BDNF in Cisd2.sup.−/− Mice

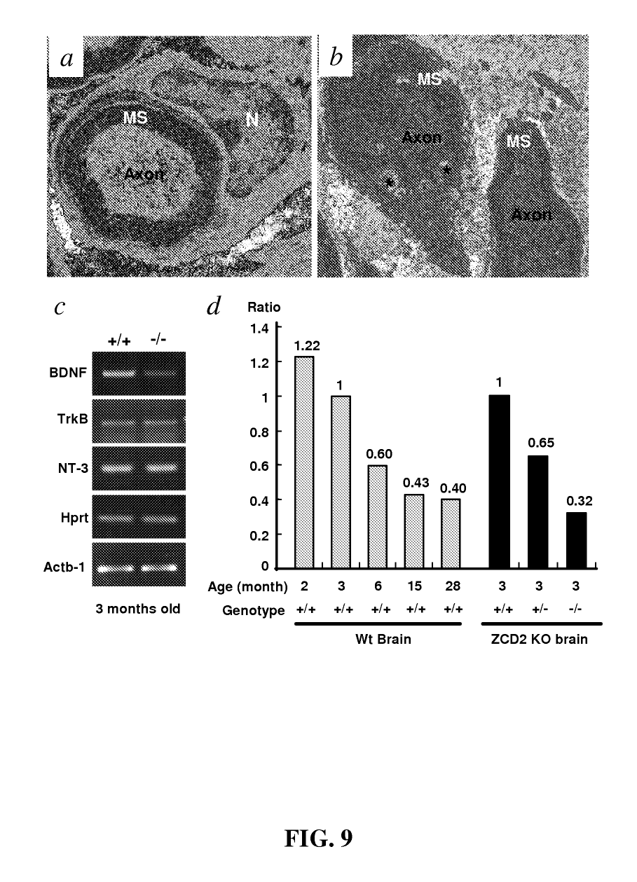

Since myelin sheath degeneration one of the clinical feature in aging, we sought to examine the state of peripheral nerves when Cisd2 gene expression is eliminated. Remarkably, there appeared to be considerable demyelination occurring in Cisd2.sup.−/− nerves with apparent axonal degeneration when ultrastructure of axons and their myelin sheath were examined (FIG. 9b).

To investigate the effect of Cisd2 on transcription of other genes, select number of genes was examined. Reverse transcription was performed with 2 μg of total RNA and primed with random hexamers and Superscript III reverse transcriptase (Invitrogen Life Technologies). Real-time PCR was carried out on Roche LightCycler 480 Real-time PCR instrument, using TaqMan probe searched at Universal ProbeLibrary (Roche applied science) and LightCycler TaqMan Master (Roche applied science). Cycling profiles for real-time PCR were pre-incubated for 10 sec at 95° C., and carried out 55 cycles of 5 sec at 95° C., 20 sec at 60° C., and 2 sec at 72° C. Fluoresce was acquired on each elongation step during amplification and analyzed with the Light Cycler Software 4.05. Significantly, brain-derived neurotrophin factor gene (BDNF) was found to be down-regulated while the levels of other genes such as TrkB, NT-3, HRPT, and Actb-1 remain unchanged. This correlates with the observation that expression levels of BDNF decrease with age as demonstrated in the 15-month and 28-month-old wild-type mice compared with that in the younger mice (FIG. 9d). Dose-dependent decrease of BDNF mRNA was detected in the heterozygous and homozygous Cisd2 knockout mice. Notably, the expression level of BDNF in the brain of 3-month old homozygous mice is lower than that in the brain of 28-month old wild-type mice. Further to the phenotypes described above, myelin sheath and axon degeneration together with down-regulation of BDNF expression add to the spectrum of premature aging features in Cisd2.sup.−/− mice.

Insofar as the description above and the accompanying drawing disclose any additional subject matter that is not within the scope of the single claim below, the inventions are not dedicated to the public and the right to file one or more applications to claim additional inventions is reserved.

Although a very narrow claim is presented herein, it should be recognized the scope of this invention is much broader than presented by the claim. It is intended that broader claims will be submitted in an application that claims the benefit of priority from this application.

What is claimed is:

1. A knockout mouse whose genome is disrupted by an inserting sequence or mutant at Cisd2 gene locus so as to produce a phenotype, relative to a wild-type phenotype, comprising aging of said knockout mouse.

2. The mouse of claim 1, wherein the loss of all or a part of the Cisd2 gene function is due to a disruption of the Cisd2 gene.

3. The mouse of claim 1, wherein the phenotype of aging comprising reduced life span, growth retardation, prominent eyes, protruding ears, scalp alopecia, early depigmentation in the fur, ocular abnormalities, reduced dermal thickness, reduced subcutaneous adipose layer, reduced thickness of muscle, increase thickness of dermis layer, decrease in femur density, lordokyphosis, reduced thoracic volume, abnormal pulmonary functions, muscle atrophy, loss of body fat, degenerated myofilaments in muscle fibers, myelin sheath degeneration, axon degeneration, or reduction of brain BDNF expression.

4. The mouse of claim 1, wherein the disruption occurs in Cisd2 exon 3.

5. The mouse of claim 1, wherein said Cisd2 gene is disrupted by recombination with homologous nucleotide sequence.

6. The mouse of claim 1, wherein the insert sequence comprising
(a) an additional copy of a Cisd2 gene fragment consisting of a portion of intron 1, the entire exon 2, and a portion of exon 3 of the Cisd2 gene;
(b) a positive puromycin selection marker;
(c) a non-functional 3′-HPRT cassette, and
(d) a loxP site.

7. A cell or cell line which comprises a targeted disruption in Cisd2 gene in which Cisd2 exon 3 has been disrupted.

8. The cell or cell line of claim 7, which is an undifferentiated cell.

9. The cell or cell line of claim 8, wherein the undifferentiated cell is selected from the group consisting of: a stem cell, embryonic stem cell oocyte and embryonic cell.

10. A method for screening a candidate agent for preventing or treating aging associated phenotypes or delaying onset of aging comprising:
(a) providing the mouse of claim 1;
(b) adding to said candidate agent, and
(c) determining the agent by identifying the desired therapeutic effects in ameliorating aging associated phenotype.

11. The method of claim 10, wherein the aging associated phenotype comprising reduced life span, growth retardation, prominent eyes, protruding ears, scalp alopecia, early depigmentation in the fur, ocular abnormalities, reduced dermal thickness, reduced subcutaneous adipose layer, reduced thickness of muscle, increase thickness of dermis layer, decrease in femur density, lordokyphosis, reduced thoracic volume, abnormal pulmonary functions, muscle atrophy, loss of body fat, degenerated myofilaments in muscle fibers, myelin sheath degeneration, axon degeneration, and reduction of brain BDNF expression.

12. The method of claim 10, wherein the agent is a test compound.

13. The method of claim 12, wherein the identification involves
(a) contacting a target gene having altered expression in a mutant Cisd2 mouse with a test compound;
(b) determining expression of said target gene, and
(c) identifying a compound that modulates expression of said target gene to a level of expression consistent with a wild type level of expression.

14. The method of claim 12, wherein the test compound is substance, molecule, compound, mixture of molecules or compounds, or any other composition which is suspected of being capable of restoring an expression level of a target gene to a more normal level.

15. A method for screening a candidate agent for preventing or treating aging associated phenotypes or delaying onset of aging comprising:
(a) providing the cell or cell line of claim 7;
(b) adding to said candidate agent, and
(c) determining the agent by identifying the desired therapeutic effects in ameliorating aging associated phenotype.


SEQUENCE LISTING (5 sequences)

SEQ ID NO: 1 - DNA, 2881 bp, Mus musculus
  gene (1)..(2881)
ccgcgagcccgccggccgcttccgctctcacgcaagcgcagcagccgggcagcggacgccgccggcggcaggatggtcctggacagcgtggcccgcatcgtgaaggtgcagctgcccgcctacctcaagcagctcccggtccccgacagcatcaccgggttcgcccgcctcacagtttcagactggctccgcctactgcccttcctcggggtacttgcgcttctgggctacctcgcagtgcgcccattcttcccaaagaagaagcaacagaaggatagcttgatcaatcttaagatacaaaaggaaaatcccaaggtggtgaatgagataaacattgaagatctgtgtctcaccaaagcagcttattgtaggtgctggcggtccaagacgtttcctgcctgtgatggatcccataataagcataatgaattgacaggcgataacgtgggtcctctcatcctgaagaagaaagaagtatagcagtggccatttgagatgaaactctgacagagtgtttccatgtgtgttgtgcatagaaaagtctttggaagttttaactctcagtactggttgaataattatttctgccagttattttcttgctacactactgcttatatttggtactttatatagtcagtcatttctatagaaatcagatggtcaagtctcattctgaaattaagtttaatgtgtctttcaacaataaagtcagcacttcagattttaattaattgggaatttaaagtttgactgtgggccaaaagttatttctttgtatatcagtatttcaacagaatacatagttttaaaaaaattatcctttgttctgttaaatgtatctttatattgtcttgttttgctcatttgaagtcatttgccatttttttggccttttgtggaactctgctgcttttagaagaattatgaaaaataattgccatcaacaatattttgaggcagtaaaaccccttgttttatagagactttcatcttattaccatgattatcccaatttctaaaaagtaaatcagatataaaaatttccaattagaaacaattattttcatcataaaaatggttcaccttacaaaagccttgttgtgtgtgtgagggtgtgagggtgtatatgtgtgtgtgagtatgtgtgtgtgtgtgagagagagagagtatgtgtgtgtgtgtgtggtgcttaacttggagactgctgggtggcaagagcactcatgaactagagtttgaagatcagttagatattaaataactgaatttgcattaaaatgatgcatagttcattgctcaaaattatgttttatcttttctgttgtaaattatagagaaacggcaacaggatattaagttcttgtattctaatggattgaatttgtacatactgagagaaaatattttgatcttttttaaaagagataactcttgttgtatttttttaacgtcacaaaatatagatcaaacacttgtgggcataatgacagaaaaaaagtctggaaggaccaaggactatgactatgacacagcatggtccacttgcacggataaggacgaggacttagcaaaggttgctgcttttactctctatttctcttccccccccccccccccgcaacaaacctttatatgctttgcttttaattgatcttttcttaattgaggctttgaatagaatcgtcatagcacagttaagctctttaggcccgagtgataagacctgattgattgtctaaggatagggtggagccagcagggcaggaagcatcatcacaagtggattagacagaggctgttcgaggagaggaagccacacacgtgttacagcaaatactgaaaaaaccaaaacaccaacagtcctttcacgtctggctcctaagtgaaatgacacgtcaagggcaaggtaggctaaataaagaaggtgggctttaagtatgtacttcagttctgccgtgttcccccagaagcagggtaaaagcaagacagttaaatctggggacactcacctgtctgcctgctggagtagatggaccatctctaggatttcttctttattaaatgagcacacagtatgcctggtaccacatgaattctgtttgttctctttctttagctatcctatctagcagtttgagaaagactagtgatggcaagctaaaaaggaaaatgattaccagtcaacttgtcacaaaattaagttacactgacacagtttgtagtcttagtttgtgtattgtaaagtgtaaataatgttaaaacagacagacatgttttagaaagacttatttcatgtgtatgagtgcctgtctgcctgtctgtgtgtctgtctgtgcaccatgcacatgcctggtgcccgaggaagccagagagaatgttggatccctggaactggaattaacagatagttgtgagctaccatgtgaatactgggaactgaacctggtctcctacaagaacatccagtgctattaacaactgagccatctctctaaccctataaaacttttaataaaagaggttatgattggtttgaagtagcaactgaaatgtggaaggaagtattccgaaaggcagaagtggtacaaagccgtgtctgctcactccactcatggtactgaggatggaacccagggcctcccacaccctggcaagagcccttcacactgagctccgtcttgagttgtatcttttcaatagcataaaccattgagacaaaatacaagaaagttttccatataggtttactggaatttttgaattatttgtgattttagcctactatcagaagtctctttagttttattttacaagtctcatgtaagagctattcttaaactattaaagatacaacctactcgttaaaaaaaaaaaaaaa

SEQ ID NO: 2 - DNA, 19 bp, Artificial
  primer
  misc_feature (1)..(19)
acttgcgcttctgggctac

SEQ ID NO: 3 - DNA, 20 bp, Artificial
  primer
  misc_feature (1)..(20)
ccagcacctacaataagctg

SEQ ID NO: 4 - DNA, 21 bp, Artificial
  primer
  misc_feature (1)..(21)
gaattgacaggcgataacgtg

SEQ ID NO: 5 - DNA, 21 bp, Artificial
  primer
  misc_feature (1)..(21)
tataagcagtagtgtagcaag